(19) United States
(12) Patent Application Publication
(10) Pub. No.: US 2026/0083526 A1
(43) Pub. Date: Mar. 26, 2026

(54) SURGICAL SPLASH SHIELD FOR USE DURING SURGICAL PROCEDURES

(71) Applicant: IJT Surgical, LLC, South Glastonbury, CT (US)

(72) Inventors: Ian J. Thomas, South Glastonbury, CT (US); Colin Thomas, South Glastonbury, CT (US)

(21) Appl. No.: 19/338,175

(22) Filed: Sep. 24, 2025

Related U.S. Application Data
(60) Provisional application No. 63/699,355, filed on Sep. 26, 2024.

(51) Int. Cl. A61B 90/00

(52) U.S. Cl. CPC A61B 90/05

(57) ABSTRACT

A splash shield either alone or in combination with a surgical device, such as a cup impactor, is provided to impede the egress of splashing blood, debris, and fluid from contacting surgeons, operating room personnel, and operating room infrastructure during surgery. The splash shield has a blocking surface with a center aperture capable of slidable, attaching movement along a shaft of the cup impactor handle or other surgical instrument. The splash shield has an outer perimeter that is greater than the surgical device shaft's outer perimeter. The center aperture has a resilient inner diameter for forming a fluid-tight, slidable grip with the shaft, or may have a slotted groove for receiving the surgical device. The splash shield can be attached to the surgical device by a clamp.

### BACKGROUND OF THE INVENTION

### 1. Field of the Invention

A surgical splash shield is presented for use during surgery, including use on automated or manual general impaction devices, such as but not limited to surgical procedures for Total Hip Arthroplasty, total knee arthroplasty, and other general orthopedic surgeries, diverting splashing blood, debris, and other fluids from contacting the surgeons, surgical staff, nurses, and/or other operating room personnel. The surgical splash shield is used in combination with devices used in these procedures that may cause surgical splatter during surgery. The proper use of acetabular cup impactors, acetabular liner impactors, femoral broaches, automatic impactors, and/or tibia punches, to name a few, is capable of splashing blood during operation, insomuch as their application requires deliberate movement of the device against the patient, and specifically against the incision location. The surgical splash shield is designed to be slidably positioned along a device to impede the egress of splashing fluids from the operating environment. It may also be placed over a shaft of a surgical device or claimed thereto.

### 2. Description of Related Art

It is not uncommon for blood, debris, and other fluids to splash or splatter during surgery. It is actually a normal part of the surgical process. When surgeons make an incision, blood vessels can release pressurized blood. As the blood flows out of the vessel, sometimes in a spraying fashion, it can come into contact with anything in the operating room, including the surgeon's or nurse's hands, eyes, face, hair, and shoes, as well as the operating room environs. This problem has presented itself numerous times, so much so that the Centers for Disease Control and Prevention (CDC) has recommended a set of standard precautions that should be taken when handling any bodily fluid, including using barriers for protection, such as surgical drapes and covers and in part why surgical staff wears personal protective equipment during surgery such as gowns, gloves, masks and eye protection.

An intra-operative splash is a common occurrence in Orthopedic surgery especially elective knee and hip replacement surgery and can potentially transmit bloodborne diseases with devastating consequences. Transmissible blood-borne infection can occur at muco cutaneous membranes. During trauma and orthopedic surgery, the use of power tools, hammers, and other force insertions increases the spraying and splattering of bodily fluids, hence resulting in an increased risk of infectious splash injury to those in the operating room. Moreover, it has been shown that the face is vulnerable to material and fluid strikes during joint arthroplasty surgery.

Universal precaution measures including personal protective equipment (PPE), such as gloves, gowns, protective eyewear, face shields, and other protective barriers, are intended to prevent parenteral, mucous membrane, and intact skin exposure to bloodborne pathogens. However, this personal protective equipment will only protect the wearer, and not prevent any spray that spreads beyond the person, to other supporting personnel, the operating equipment, and/or accompanying medical devices, the operating floor, lights, ceiling, and other environs.

As an illustrious example, in a total hip replacement procedure (also called total hip arthroplasty), the damaged bone and cartilage is removed and replaced with prosthetic components. In the process of performing a hip replacement, spurious blood and bodily fluid spray is not uncommon. FIG. 1A depicts an exploded view of a hip replacement implant device 10, showing the individual parts of the hip replacement prosthesis. Typically, the damaged femoral head is removed and replaced with a metal femoral stem 12 that is placed into the hollow center of the femur. The femoral stem may be either cemented or “press fit” into the bone. A ball 14 (typically metal or ceramic) is placed on the upper part of the stem. Ball 14 replaces the damaged femoral head that was removed. The damaged cartilage surface of the socket (acetabulum) is removed and replaced with a metal socket or acetabular component 16. Screws or cement are sometimes used to hold the socket in place. A plastic, ceramic, or metal spacer 18 (also referred to as a liner) is inserted between the new ball and the socket to allow for a smooth gliding surface. FIG. 1B depicts the assembled hip replacement implant 10. Although a total hip arthroplasty procedure is discussed as an application for the surgical splash shield of the present invention, other procedures may also require the need for splash mitigation, such as total knee arthroplasty, and other orthopedic procedures, and the application of a splash shield on surgical devices for these other procedures would serve the same protective purpose.

A hip arthroplasty is a procedure designed to directly change the contour of, or to replace, the hip joint (acetabulum) or femoral head. This term, hip arthroplasty, is so general that it includes most procedures for developmental dysplasia of the hips (DDH), osteoarthritis, and all procedures that involve prosthetic replacement.

An issue in total hip arthroplasty, total knee arthroplasty, and other general orthopedic surgeries, is the splashing and splattering of blood, debris and other fluids during surgery. As noted above, protective measures have been developed to limit the exposure of the surgeons and nurses utilizing facial and body coverings; however, little has been done in a manner that would divert the splashing and spattering closer to the source; that is, locating a splash shield or protector closer to the patient and surgical site, while allowing the surgeon and other operating room personnel to work around the shield without degradation of the procedure.

### SUMMARY OF THE INVENTION

Bearing in mind the problems and deficiencies of the prior art, it is therefore an object of the present invention to provide a splash shield for application in total hip arthroplasty (“THA”), total knee arthroplasty (“TKA”), and other general orthopedic surgeries, which can be attachable to the applicable surgical device, such as for example, a cup impact handle for a THA application, for diverting splashing blood, debris, and other fluids from contacting the operating room staff and surrounding operating room infrastructure during surgery. The splash shield may also be adjustable on the surgical device.

It is another object of the present invention to provide an adjustable splash shield having a universal attachment to a surgical device, such as a cup impact handle or offset impactor device, where the surgical device includes a shaft component, and the splash shield includes an extended blocking surface having a center aperture for slidable positioning along the surgical device. For example, located on the shaft of the cup impact handle, the center aperture may have an aperture with a resilient inner periphery for resilient, secure attachment with the shaft, allowing the splash shield to be slidably placed close to a point of contact with a patient during surgery, and generally proximate to the incision point.

It is yet another object of the present invention to provide a splash shield that is capable of attaching to an acetabular cup impactor, an acetabular liner impactor, broach handle, automatic impactor, or a tibia punch, to divert splashing blood, debris, and fluids. The splash shield being slidably attachable to a shaft of the cup impactors, and having a shape that blocks and/or diverts the spraying fluid away from the operating room staff and operating room infrastructure. The splash shield/surgical device attachment may be in the form of a resilient friction fit, a clamp, a slotted groove, or other attachment schemes capable of blocking fluids during operation.

In a first aspect, the present invention is directed to a combination splash shield and surgical device for diverting splashing blood, debris, and fluid from contacting the surgical staff, operating room personnel, and/or operating room infrastructure during surgery, comprising: the surgical device having a handle connected to an elongated shaft; the splash shield including an extended blocking surface and a center aperture for attachment to the elongated shaft of the handle, the center aperture having a resilient inner diameter for slidable contact with the shaft, allowing the blocking surface to be placed in close proximity to an incision point on a patient during the surgery; the blocking surface having an outer perimeter that extends beyond an outer perimeter of the shaft, such that the blocking structure located between a user and the patient is capable of shielding and blocking splashing blood, debris, and other fluids.

The splash shield may be removably attachable to a plurality of different handles or shafts, and can be fabricated to be attached on such surgical instructions as an acetabular cup impactor, an acetabular liner impactor, femoral broach handle, automatic impactor, or a tibia punch.

The splash shield may be slidably movable in a longitudinal direction along the shaft or the handle, having a cross-section of the center aperture which is sized and shaped for different tools that have shafts with different cross-sectional shapes.

The combination includes the center aperture producing a tight, slidable fit with the shaft such that it can be placed in one position or another on the shaft, and physically hold itself in place in a friction fit caused by a tightened elasticity of the resilient inner diameter against the outer perimeter of the shaft.

The splash shield blocking surface may form curved or folded portions extending from the center aperture to direct any splashing inwards.

The center aperture may comprise a circular, oval, rectangular, square or other quadrilateral, triangular, or polygon cross-sectional shape.

Furthermore, the splash shied may include a radial slot for insertion over the surgical device, and may include material covering the radial slot to block fluid from traversing through the radial slot.

The splash shield may include a clamp for attachment to the surgical device.

In a second aspect, the present invention is directed to a splash shield for diverting splashing blood and other body fluids from contacting the surgical staff, operating room personnel, and/or operating room infrastructure during surgery, comprising: an extended blocking surface having a center aperture for attachment to a shaft or handle of a surgical device, the center aperture having a shape matching a cross-sectional surface of the shaft for peripheral contact therewith, allowing the blocking surface to be placed close to a point of contact with a patient during the surgery; the blocking structure having an outer perimeter that extends beyond an outer perimeter of the shaft, such that the blocking surface located between a user and the patient is capable of shielding the user from the splashing blood and other body fluids.

The center aperture is designed to effect a tight, slidable fit with the shaft or handle such that the splash shield can be placed in different positions, and physically hold in place in a friction fit caused by a tightened elasticity of a resilient inner surface of the center aperture against the outer perimeter of the shaft.

The blocking surface may be formed of thermoplastic films including Polyester (PET), Polycarbonate (PC), or (PETG), specialty acrylic, high-density polyethylene, kydex thermoplastic, and/or acrylonitrile butadiene styrene.

The blocking surface may be fabricated from a light, shapable metal, having material sufficiently rigid to hold its shape against a bodily fluid spraying force.

The blocking structure may include stainless steel, copper, titanium, cobalt chrome, aluminum, magnesium, gold, platinum, silver, iridium, and/or tantalum, and composites thereof.

The center aperture may comprise a circular, oval, rectangular, square or other quadrilateral, triangular, or polygon cross-sectional shape.

### BRIEF DESCRIPTION OF THE DRAWINGS

The features of the invention believed to be novel and the elements characteristic of the invention are set forth with particularity in the appended claims. The figures are for illustration purposes only and are not drawn to scale. The invention itself, however, both as to organization and method of operation, may best be understood by reference to the detailed description which follows taken in conjunction with the accompanying drawings in which:

FIG. 1A depicts an exploded view of a hip replacement implant, showing the individual parts of the hip replacement prosthesis;

FIG. 1B depicts the assembled hip replacement implant of FIG. 1A;

FIG. 2 depicts an acetabular cup impactor with an attached metal socket or acetabular component, and an elongated shaft having a slidable splash shield attached thereto;

FIG. 3 depicts a side view of the splash shield of FIG. 2, showing one embodiment having preferred dimensions;

FIG. 4 depicts an offset cup impactor utilizing a splash shied proximate the acetabular component;

FIG. 5 depicts the acetabular cup impactor of FIG. 2 with the acetabular component removed from the shaft and the splash shield slidably located closer to the patient and consequently incision point;

FIG. 6 depicts another embodiment of an acetabular cup impactor having a flat disc-shaped splash shield with a circumferential flange end extending towards the incision cut;

FIG. 7 depicts a femoral broach handle with an attachable/releasable disc-shaped splash shield, where in the embodiment shown, the splash shield includes a center aperture that corresponds to the periphery cross-sectional shape of the femoral broach handle, and a separation slot for attachment on handle at any desirable point along the handle shaft;

FIG. 8A depicts a partial domed-shaped splash shield having a center aperture for shaft insertion;

FIG. 8B is a bottom side view of the partial domed-shaped splash shield of FIG. 8A delineating the different diameters of salient dimensions; and

FIG. 8C is a cross-sectional view of the partial dome splash shield of FIG. 8A, depicting a height dimension and radius of curvature for a desired dome shape.

### DESCRIPTION OF THE PREFERRED EMBODIMENT(S)

Embodiments of the present invention now will be described more fully hereinafter with reference to the accompanying drawings, in which embodiments of the invention are shown. This invention may, however, be embodied in many different forms and suitable for many different surgical devices and procedures, and should not be construed as limited to the embodiments set forth herein. Rather, these embodiments are provided so that this disclosure will be thorough and complete, and will fully convey the scope of the invention to those skilled in the art. Like numbers refer to like elements throughout.

It will be understood that, although the terms first, second, etc., may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the singular forms “a”, “an” and “the” are intended to include the plural forms as well, unless the context clearly indicates otherwise. Also, as used herein, the term “and/or” includes any and all combinations of one or more of the associated listed items. It will be further understood that the terms “include”, “including”, “comprise”, and/or “comprising” when used herein, specify the presence of stated features, steps, operations, elements, and/or components, but do not preclude the presence or addition of one or more other features, steps, operations, elements, components, and/or groups thereof.

It will be further understood that when an element is referred to as being “on” or extending “onto” another element, it can be directly on or extend directly onto the other element or intervening elements may also be present. In contrast, when an element is referred to as being “directly on” or extending “directly onto” another element, there are no intervening elements present. It will also be understood that when an element is referred to as being “connected,” “coupled”, “operatively coupled”, or the like to another element, it can be directly connected, coupled, or operatively coupled to the other element or intervening elements may be present. Moreover, it can be removable or integral with the other element and/or intervening elements. In contrast, when an element is referred to as being “directly connected”, “directly coupled”, or “directly operatively coupled” to another element, there are no intervening elements present.

Relative terms such as “below,” “above,” “upper,” “lower,” “horizontal,” “vertical,” “top,” “bottom,” “rear,” “front,” “side,” or the like may be used herein to describe a relationship of one element or component to another element or component as illustrated in the figures. It will be understood that these terms are intended to encompass different orientations of the device in addition to the orientation depicted in the figures.

Additionally, in the subject description, the words “exemplary,” “illustrative,” or the like are used to mean serving as an example, instance or illustration. Any aspect or design described herein as “exemplary” or “illustrative” is not necessarily intended to be construed as preferred or advantageous over other aspects or design. Rather, use of the words “exemplary” or “illustrative” is merely intended to present concepts in a concrete fashion.

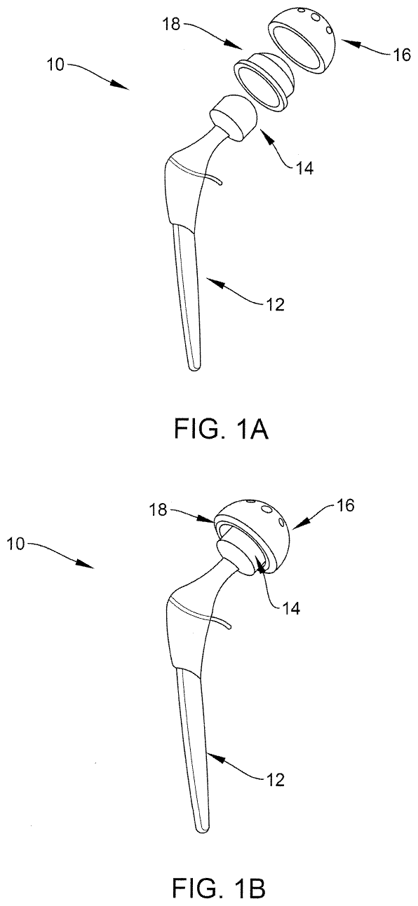

In describing the preferred embodiment of the present invention, reference will be made herein to FIG. 1-8 of the drawings in which like numerals refer to like features of the invention.

Total hip replacement surgery is described herein as an exemplary embodiment; however, other surgical procedures are not precluded, and the splash shield can be readily adapted for different surgical devices and procedures.

Total hip replacement surgery is a surgical procedure whereby the bones which make up the hip joint are replaced with prosthetic implants. The stability of acetabular cup implants, as part of a total hip replacement (THR), is a major influencing factor in terms of the overall long-term survivorship of the implant. However, in surgical procedures such as this, splashing of blood, debris, and fluids are not uncommon, and droplets of blood or aerosolized blood may be exposed to the surgeon, surgical staff, nurses, and other operating room personnel. This splashing and splattering can detrimentally affect the surgical procedure, by exposing surgical personnel to a hazardous environment, altering visibility, and diverting attention, among other constraints to the operation. It has been found that redirecting the fluids from a point closer to the patient would minimize the amount of splash that could reach those performing and/or assisting the surgery.

In an exemplary embodiment of the present invention, a splash shield or protector is utilized in hip replacement surgery during acetabular cup impaction. (Similar application to total knee arthroplasty (TKA) and other orthopedic procedures may also be accomplished by the implementation of this invention.) The splash shield is designed so that it is removably attachable to a plurality of different cup impaction handles and other surgical devices. As an example, the surgical splash shield may be situated on an acetabular cup impactor, an acetabular liner impactor, femoral broach handle, an offset impactor, a tibia punch, or an automatic impactor, to name a few surgical tools that would benefit from the application of a splash shield. As an illustrative example, the splash shield or protector is attached to the shaft of an acetabular cup impactor such that the splash shield is capable of blocking blood, debris, and fluid spray from the patient close to the point of use.

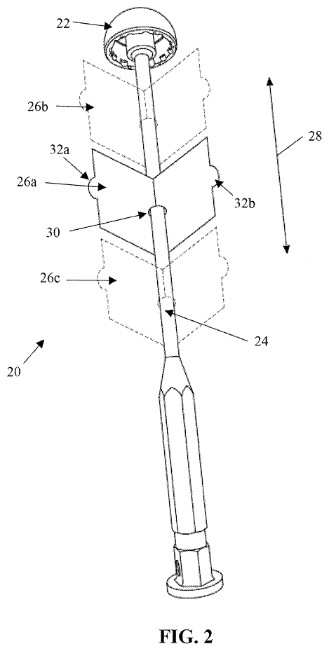

FIG. 2 depicts an acetabular cup impactor 20 with an attached metal socket or acetabular component 22, and an elongated shaft 24. A version of the splash shield is seen attached over the shaft of the impactor. A splash shield 26a is shown slidably attached to shaft 24. Motion of splash shield 26a is in the longitudinal direction along shaft 24, as illustrated by arrow 28. Phantom, dash-lined FIGS. 26b,c shown in FIG. 2 indicate illustrious examples for the various slidable, longitudinal placement positions of the splash shield. Splash shield 26a may be positioned closer to the patient and the incision point, or further from the patient along the shaft, wherever the attending surgeon desires.

Splash shield 26a provides a blocking surface extending outwards from the shaft, forming a blocking structure, having a center aperture 30 sized to receive the outer perimeter of shaft 24 circumferentially. Although shown as a circular aperture in this embodiment, the center aperture 30 of splash shield 26a may be sized and shaped for different tools that have shafts with different outer contours that are not simply circular in cross-section. The center aperture is designed to be sufficiently resilient to produce a fluid-tight, slidable fit with the shaft so that it can be movably (slidably) placed in one position or another on the shaft, and physically hold itself in place in a friction fit caused by the tightened elasticity of its material against the shaft perimeter. In one embodiment, a resilient o-ring fitted to the inside of the aperture may be employed to establish a frictional fit to the shaft. This tight fit is generally due to the resilient material make-up of the peripheral edge of the center aperture. The tight, friction fit of the shield on the shaft will prohibit blood, debris, and fluids from traversing through the center aperture, but is capable of allowing for slidable interaction with the shaft itself.

In the embodiment of FIG. 2, splash shield 26a is contoured such that it is capable of covering a majority of the incision footprint while providing sufficient visibility for the surgeon. Splash shield 26a may include curved (or folded) portions extending from the center aperture 30 to direct any splashing inwards, and in some instances, back towards the incision location. Body contour tabs 32a,b may be added, and can be located at the distal end of each curved (or folded) portion, and may be used to place the splash shield in close proximity to the incision point to shield and redirect the splashing. The contour tabs allow for gripping and facilitating movement of the splash shield in-situ during operation if desired.

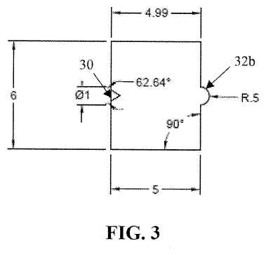

FIG. 3 depicts a dimensional side view of an exemplary splash shield, where exemplary dimensions are illustrated. Body contour tab 32b is shown having a predetermined radius of curvature (R.5) at one end of the splash shield, and a triangular shaped center aperture 30 at an opposite end. In this particular case, a diamond-shaped center aperture is formed with approximately a sixty-two degree (62°) cut from the side surface in order to tightly fit against a shaft but allow for slidable movement against a shaft having a quadrilateral cross-section. The diamond shaped aperture is depicted as a triangular cut in the one-sided view shown in FIG. 3. It is also possible to secure the splash shield directly to the shaft, making a fixed combination upon assembly, if a permanent position is deemed more suitable over others. Such an attachment may be in the form of a clamp, securing the splash shield to the shaft at a given location.

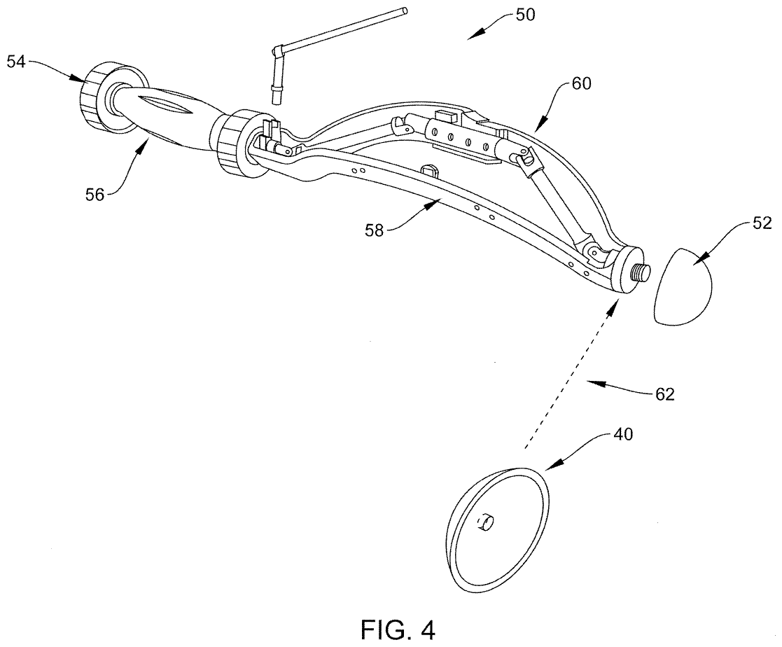

FIG. 4 depicts an offset cup impactor 50 utilizing a splash shied 40 proximate the acetabular component 52. The offset cup impactor is a reusable handheld instrument designed for use by orthopedic surgeons specifically for the purpose of inserting and implanting acetabular cup components during total hip arthroplasty (THA) surgery. The impactor provides a means for holding, inserting, and impacting an acetabular cup implant and may include an impact sleeve designed to protect the mating implant interface. Generally, an offset cup impactor includes an anvil 54 attached at one end to a handle 56. The handle 56 is attaches to a shaft body 58 and actuator arm 60.

In another application, the splash shield may be combined with an automatic impactor. An automatic impactor is an advanced surgical device, primarily used in total hip arthroplasty, designed to deliver consistent and controlled force during surgical steps like femoral broaching, rather than relying on manual hammering by the surgeon. Automated impactors have emerged as innovative tools to address the limitations of mallets, with efforts to improve the surgeon's physical health, efficiency, and patient outcomes. The implementation of a splash shield with an automatic impactor would reduce the backlash of fluid splashing and spraying in a similar fashion as the application of a splash shield to a non-automatic impactor.

The dimensions of splash shield 40 may vary depending upon the surgical instrument used. For example, the radius of splash shield 40 may be greater for an offset impactor to extend radially outwards beyond, and thereby cover, the radial extension of the offset cup impactor actuator arm 60. Dashed line 62 indicates the placement region of splash shield 40 on the surgical device.

The splash shied may be formed of thermoplastic films including Polyester (PET), Polycarbonate (PC), or (PETG), or more flexible plastics, such as specialty acrylic, high-density polyethylene, kydex thermoplastic, or acrylonitrile butadiene styrene, to name a few. The splash shield may or may not be transparent, and if semi-opaque, could be fabricated from a light, shapeable metal, having material sufficiently rigid to hold its shape and withstand any spraying force, and to the extent it may be reusable, capable of facilitating sterilization between uses, such as stainless steel, copper, titanium, cobalt chrome, aluminum, magnesium, gold, platinum, silver, iridium, tantalum, or any combination thereof, and the like. The center portion of the splash shield that interfaces about the periphery of the outside surface of the shaft may be made of a resilient material that is distinctly different from the remainder of the splash shield, and is capable of providing a resilient, frictional force to the shaft on an inner side, while providing a resilient attachment to the shield on an outer side.

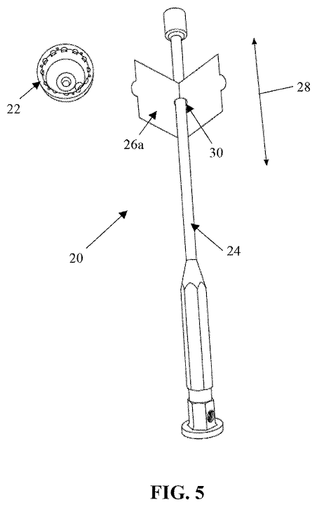

FIG. 5 depicts the acetabular cup impactor 20 of FIG. 2 with the acetabular component 22 removed from the shaft 24, and the splash shield slidably located closer to the patient and incision point. Center aperture 30 may be a cutout at the center of the splash shield, or may include a resilient component or ring of a material different from the blocking surfaces of the splash shield, and situated circumferentially about the aperture inside edge for the purpose of forming a flexible press-fit against shaft 24. In one embodiment, the resilient material at the center is sufficiently flexible to be placed over the end of the impactor, but still retain elasticity to form a tight fit on the shaft. The splash shield may be easier to slide along the shaft, and still provide an impervious stop for fluids splashing away from the incision, towards the surgeon, supporting staff, surgical equipment, and other structures in the operating room. The fluid-tight, resilient, inner aperture of the splash shield, which for a circular cross-section may be a slidable ring, need not be the same material as the splash shield blocking structure itself. For a circular cross-section example, the ring may form a slidable O-ring type seal about the shaft on the inside surface, while having a form fit shaped surface on the outside for abutment to the splash shield receiving center aperture.

Application of a slidable splash shield to a tibia punch may be performed in a similar manner to the attachment to the shaft of an acetabular cup impactor.

Regardless of the radial, cross-sectional shape of the shaft (circular, oval, quadrilateral, (rectangular, square), triangular, polygon, etc.), the center aperture of the splash shield is shaped accordingly, such that a tight, press-fit is achieved for slidable, fluid-impervious interaction. In this manner, the present invention is not limited to any particular shaft cross-sectional shape, and may be adaptable for different cross-sectional shaft shapes of various surgical devices.

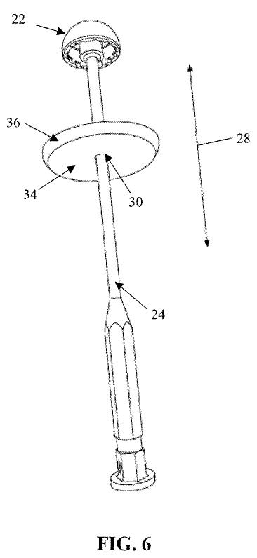

FIG. 6 depicts the acetabular cup impactor 20 of FIG. 2 with a flat disc-shaped splash shield 34 having a circumferential flange end 36 extending towards the incision cut. The flange may be angled away from, or perpendicular with, the radial extension of the flat disc of the blocking structure. Center aperture 30 may be designed as discussed above for this and other shaft cross-sectional shapes.

Alternatively, in instances where the surgical instrument is not geometrically designed for slidable movement of a splash shield, such as in the application of a femoral broach handle, such instruments may require an attachable/releasable splash shield, which may include a splash shield fabricated of material that incorporates a slot or cut for separation of the splash shield, allowing the user to insert the splash shield around the handle, and either through material resilience or an attachment component, seal the splash shield about the handle.

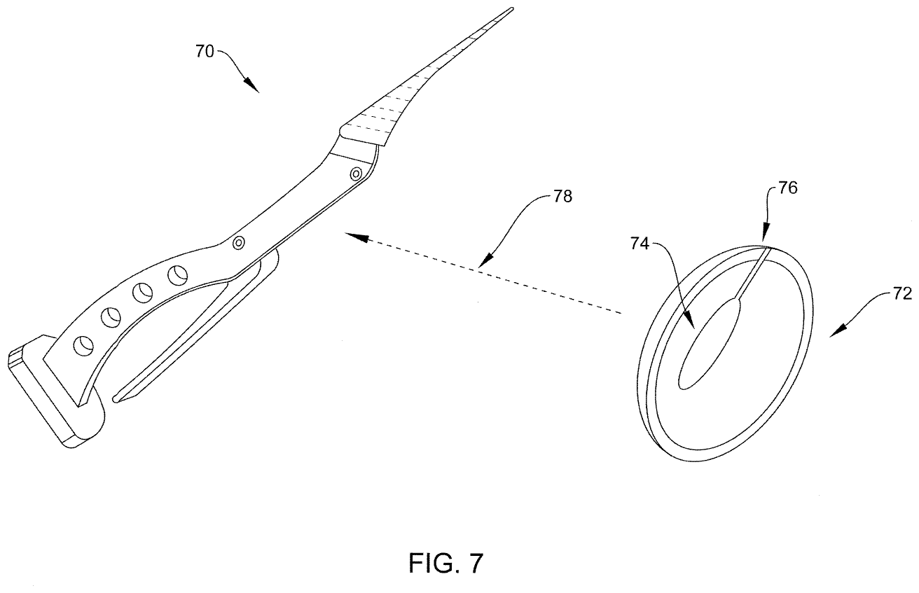

FIG. 7 depicts a femoral broach handle 70 with an attachable/releasable disc-shaped splash shield 72, where in the embodiment shown, the splash shield includes a center aperture 74 that corresponds to the periphery cross-sectional shape of the femoral broach handle, and a separation slot 76 for attachment on handle 70 at any desirable point along the handle shaft. Arrow 78 indicates a placement location for splash shield 72. The upper surface of the splash shield, the surface facing the surgeon, may include additional material or a flap to “seal” separation slot 76 from allowing fluid to pass through the splash shield. In other embodiments, a divided splash shield may be separably secured by a clamp or other attachment mechanism, and the attachment schemes for such attachments may be known in the art.

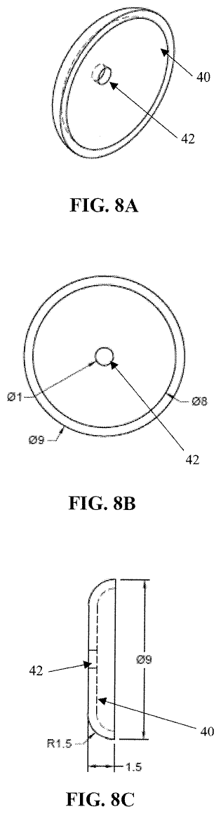

FIG. 8A depicts a partial domed-shaped splash shield 40 having a center aperture 42 for shaft insertion. The shape may be a rounded, often hemispherical form of a dome, essentially, a structure that curves upward and outward from a circular or elliptical base. FIG. 8B is a bottom side view of the partial domed-shaped splash shield 40 of FIG. 8A delineating the different diameters of salient structures. Splash shield 40 has an outermost diameter ø9 and inner diameter ø8, which may form a lip for the internal dome structure, larger than the thickness of the internal dome structure. The center aperture 42 is shown with the smallest diameter ø1. FIG. 8C is a cross-sectional view of the partial dome splash shield of FIG. 8A, depicting a height dimension and radius of curvature for the dome shape.

While the present invention has been particularly described, in conjunction with a specific preferred embodiment, it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

Thus, having described the invention, what is claimed is:

1. A combination splash shield and surgical device for diverting splashing blood, debris, and fluid from contacting the surgical staff, operating room personnel, and/or operating room infrastructure during surgery, comprising:
said surgical device having a handle connected to an elongated shaft;
said splash shield including an extended blocking surface and a center aperture for attachment to said elongated shaft of said handle, said center aperture having a resilient inner diameter for slidable contact with said shaft, allowing said blocking surface to be placed in close proximity to an incision point on a patient during said surgery;
said blocking surface having an outer perimeter that extends beyond an outer perimeter of said shaft, such that said blocking structure located between a user and said patient is capable of shielding and blocking splashing blood, debris, and other fluids.

2. The combination of claim 1 wherein said splash shield is removably attachable to a plurality of different handles or shafts.

3. The combination of claim 2, wherein said splash shield is designed to be attached on an acetabular cup impactor, an acetabular liner impactor, femoral broach handle, an automatic impactor, or a tibia punch.

4. The combination of claim 1, wherein said splash shield is slidably movable in a longitudinal direction along said shaft or said handle.

5. The combination of claim 1 wherein a cross-section of said center aperture is sized and shaped for different tools that have shafts with different cross-sectional shapes.

6. The combination of claim 1, wherein the center aperture produces a tight, slidable fit with the shaft such that it can be placed in one position or another on the shaft, and physically hold itself in place in a friction fit caused by a tightened elasticity of said resilient inner diameter against said outer perimeter of said shaft.

7. The combination of claim 1, wherein said splash shield is contoured to cover spatially a majority of an incision footprint.

8. The combination of claim 1, wherein said splash shield blocking surface forms curved or folded portions extending from said center aperture to direct any splashing inwards.

9. The combination of claim 8 wherein said blocking structure includes body contour tabs located at the distal end of said curved or folded portions, and upon sliding of said splash shield with respect to said shaft, said body contour tabs may be place in close proximity to the incision point to shield and redirect the splashing.

10. The combination of claim 9, wherein said body contour tabs include a predetermined radius of curvature at one end of the blocking structure.

11. The combination of claim 5, wherein said center aperture comprises a circular, oval, rectangular, square or other quadrilateral, triangular, or polygon cross-sectional shape.

12. The combination of claim 1 wherein said splash shied includes a radial slot for insertion over said surgical device.

13. The combination of claim 12 wherein said splash shield includes material covering said radial slot to block fluid from traversing through said radial slot.

14. The combination of claim 1 wherein said splash shield includes a clamp for attachment to said surgical device.

15. A splash shield for diverting splashing blood and other body fluids from contacting the surgical staff, operating room personnel, and/or operating room infrastructure during surgery, comprising:
an extended blocking surface having a center aperture for attachment to a shaft or handle of a surgical device, said center aperture having a shape matching a cross-sectional surface of said shaft for peripheral contact therewith, allowing said blocking surface to be placed close to a point of contact with a patient during said surgery;
said blocking structure having an outer perimeter that extends beyond an outer perimeter of said shaft, such that said blocking surface located between a user and said patient is capable of shielding said user from said splashing blood and other body fluids.

16. The splash shield of claim 15 wherein said splash shield is removably attachable to a plurality of different surgical devices.

17. The splash shield of claim 16, wherein said splash shield is designed to be situated on an acetabular cup impactor, an acetabular liner impactor, femoral broach handle, an automatic impactor, or a tibia punch.

18. The splash shield of claim 15, wherein the center aperture is designed to effect a tight, slidable fit with the shaft or handle such that said splash shield can be placed in different positions, and physically hold in place in a friction fit caused by a tightened elasticity of a resilient inner surface of said center aperture against said outer perimeter of said shaft.

19. The splash shied of claim 15, wherein said blocking surface is formed of thermoplastic films including Polyester (PET), Polycarbonate (PC), or (PETG), specialty acrylic, high-density polyethylene, kydex thermoplastic, and/or acrylonitrile butadiene styrene.

20. The splash shield of claim 15, wherein said blocking surface is fabricated from a light, shapable metal, having material sufficiently rigid to hold its shape against a bodily fluid spraying force.

21. The splash shield of claim 20, wherein said blocking structure include stainless steel, copper, titanium, cobalt chrome, aluminum, magnesium, gold, platinum, silver, iridium, and/or tantalum, and composites thereof.

22. The splash shield of claim 15, wherein said center aperture comprises a circular, oval, rectangular, square or other quadrilateral, triangular, or polygon cross-sectional shape.

23. The splash shield of claim 15 including a radial slot for insertion over said surgical device.

24. The splash shield of claim 23 including material covering said radial slot to block fluid from traversing through said radial slot.

25. The splash shield of claim 15 including a clamp for attachment to said surgical device.
